(19) United States
(12) Patent Application Publication
(10) Pub. No.: US 2026/0242725 A1
(43) Pub. Date: Aug. 20, 2026

(54) USE OF ENGINEERED MATERIALS TO LOCALLY INDUCE DIFFERENTIATION OF ENGINEERED CELLS

(71) Applicant: The Regents of the University of California, Oakland, CA (US)

(72) Inventors: Wendell A. LIM, San Francisco, CA (US); Jonathan M. BRUNGER, Nashville, TN (US)

(21) Appl. No.: 19/161,690

(22) PCT Filed: Mar. 8, 2024
(86) PCT No.: PCT/US2024/019253

§ 371 Date: Sep. 2, 2025

Related U.S. Application Data
(60) Provisional application No. 63/451,531, filed on Mar. 10, 2023.

(51) Int. Cl. C12N 5/0735; A61K 9/00; A61K 35/33; A61K 35/545; A61K 38/00; C07K 14/47; C07K 14/475; C12N 5/077

(52) U.S. Cl. CPC C12N 5/0606; A61K 9/0019; A61K 35/33; A61K 35/545; C07K 14/4702; C07K 14/475; C12N 5/0656; A61K 38/00; C12N 2506/02; C12N 2510/00; C12N 2533/14; C12N 2533/30; C12N 2533/50; C12N 2533/54

(57) ABSTRACT

Provided herein is a reagent system that may comprise a biocompatible material conjugated to a ligand and a mammalian cell comprising a molecular circuit comprising: (i) a binding activated transcriptional switch (BTTS) comprising a force-dependent protease cleavage site and a transcriptional activator and (ii) a nucleic acid encoding a payload protein. In these embodiments, binding of the cell of (b) to the ligand of (a) via the BTTS induces proteolytic cleavage of the force-dependent cleavage sites to release the transcriptional activator and induce expression of the payload protein. Examples of such circuits and their methods of use are also described.

### CROSS-REFERENCING

This application claims the benefit of U.S. provisional application Ser. No. 63/451,531, filed on Mar. 10, 2023, which application is incorporated by reference herein.

### STATEMENT REGARDING FEDERALLY SPONSORED RESEARCH

This invention was made with government support under grant nos. F32 EB024391 and P50 GM081879 awarded by The National Institutes of Health and grant number HR0011-18-2-0027 awarded by the Defense Advanced Research Projects Agency of the Department of Defense. The government has certain rights in the invention.

### BACKGROUND

Attempts to produce cell-based therapies for regeneration and repair have often failed because of the lack of an ability to direct cellular responses such as differentiation in a spatially precise way. This disclosure is believed to provide a solution to this problem.

### SUMMARY

Provided herein is a complementary material-cell pair that contains an engineered responder cell and a complementary biomaterial that is engineered to contain a ligand that is recognized by the engineered responder cells. In the example shown, the responder cells are engineered with a synthetic binding-triggered transcriptional switch (e.g., synNotch or the like), which is activated by a cognate ligand incorporated into the complementary biomaterial. The input ligands can be orthogonal to other biological interactions, thus allowing the material to only generate a response in the engineered responder cells. This type of engineered material/cell platform can be used to locally drive processes such as stem cell differentiation or the production of morphogens. This system can be used to spatially pattern cellular behaviors and organization using instructive and programmable biomaterials to which the cells have been engineered to respond to. Specifically, using this system, cells can be programmed to act locally even though they have been administered systemically.

In some embodiments, a reagent system is provided. In these embodiments, the reagent system may comprise: (a) a biocompatible material conjugated to a ligand and (b) a mammalian cell comprising a molecular circuit comprising: (i) a binding activated transcriptional switch (BTTS) comprising a force-dependent protease cleavage site and a transcriptional activator; and (ii) a nucleic acid encoding a payload protein. In these embodiments, binding of the cell of (b) to the ligand of (a) via the BTTS induces proteolytic cleavage of the force-dependent cleavage sites to release the transcriptional activator and induce expression of the payload protein. For example, in some embodiments the mammalian cell is a stem cell, the payload protein is a cell fate regulator, and expression of the payload protein causes the stem cell to differentiate.

Examples of such circuits and their uses are also described. In some embodiments, the method may comprise obtaining the system and contacting (a) and (b), thereby inducing the payload. In vivo applications may comprise: (i) administering the material to one or more sites in the mammal; and (ii) administering the cell to the mammal. In these embodiments, the cell may be administered systemically, and the payload protein will only be induced locally to the material. In some embodiments, the mammalian cell may be a stem cell, the payload protein is a cell fate regulator, and expression of the payload protein causes the stem cell to differentiate locally to the one or more sites to which the material has been administered.

### BRIEF DESCRIPTION OF THE DRAWINGS

FIG. 1 illustrates some of the principles of the present system and method.

FIG. 2 demonstrates that materials programmed with synNotch ligand activate synNotch can be used to induce morphogen (e.g. BMP4) expression in mESCs, leading to mesodermal differentiation.

FIG. 3 demonstrates that materials displaying synNotch ligand can induce neuronal differentiation of ESCs engineered to express Neurogenin2 upon synNotch activation.

FIG. 4 shows the programming of biomaterials with an anti-ligand nanobody (e.g., GFP-TRAP nanobody for capturing GFP ligand) converts the juxtacrine synNotch platform to a paracrine platform capable of detecting soluble cues.

FIG. 5: shows the ability to fine tune biomaterial binding to soluble factors.

### DEFINITIONS

As used herein, the terms “treatment,” “treating,” “treat” and the like, refer to obtaining a desired pharmacologic and/or physiologic effect and/or a response related to the treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

A “therapeutically effective amount” or “efficacious amount” refers to the amount of an agent (including biologic agents, such as cells), or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The terms “individual,” “subject,” “host,” and “patient,” used interchangeably herein, refer to a mammal, including, but not limited to, murines (e.g., rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

As used herein, the term “binding-triggered transcriptional switch” or “BTTS” refers to any polypeptide or complex of the same that is capable of transducing a specific binding event on the outside of the cell (e.g., binding of an extracellular domain of the BTTS) to activation of a recombinant promoter within the nucleus of the cell. Many BTTSs work by releasing a transcription factor that activates the promoter. In these embodiments, the BTTS is made up of one or more polypeptides that undergo proteolytic cleavage upon binding to the cognate ligand to release a gene expression regulator that activates the recombinant promoter. For example, a BTTS may comprise (i) an extracellular domain comprising the antigen binding region of an antigen-specific antibody; (ii) a proteolytically cleavable sequence comprising one or more proteolytic cleavage sites; and (iii) an intracellular domain, wherein binding of the antigen binding region to the antigen induces cleavage of the sequence at the one or more proteolytic cleavage sites, thereby releasing the intracellular domain and wherein the intracellular domain activates transcription of an expression cassette. A BTTS can be based on synNotch, A2, MESA, or force receptor, for example, although others are known or could be constructed.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

The terms “synthetic”, “chimeric” and “engineered” as used herein generally refer to artificially derived polypeptides or polypeptide encoding nucleic acids that are not naturally occurring. Synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods. Chimeric and engineered polypeptides or polypeptide encoding nucleic acids will generally be constructed by the combination, joining or fusing of two or more different polypeptides or polypeptide encoding nucleic acids or polypeptide domains or polypeptide domain encoding nucleic acids. Chimeric and engineered polypeptides or polypeptide encoding nucleic acids include where two or more polypeptide or nucleic acid “parts” that are joined are derived from different proteins (or nucleic acids that encode different proteins) as well as where the joined parts include different regions of the same protein (or nucleic acid encoding a protein) but the parts are joined in a way that does not occur naturally.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The term “operably linked” refers to a juxtaposition wherein the components so described are in a relationship permitting them to function in their intended manner. For instance, a promoter is operably linked to a coding sequence if the promoter affects its transcription or expression. Operably linked nucleic acid sequences may but need not necessarily be adjacent. For example, in some instances a coding sequence operably linked to a promoter may be adjacent to the promoter. In some instances, a coding sequence operably linked to a promoter may be separated by one or more intervening sequences, including coding and non-coding sequences. Also, in some instances, more than two sequences may be operably linked including but not limited to e.g., where two or more coding sequences are operably linked to a single promoter.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “vector” or “expression vector” is a replicon, such as plasmid, phage, virus, or cosmid, to which another DNA segment, i.e. an “insert”, may be attached so as to bring about the replication of the attached segment in a cell.

The term “heterologous”, as used herein, means a nucleotide or polypeptide sequence that is not found in the native (e.g., naturally-occurring) nucleic acid or protein, respectively. Heterologous nucleic acids or polypeptide may be derived from a different species as the organism or cell within which the nucleic acid or polypeptide is present or is expressed. Accordingly, a heterologous nucleic acids or polypeptide is generally of unlike evolutionary origin as compared to the cell or organism in which it resides.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the cell” includes reference to one or more cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As noted above, a reagent system that may comprise (a) a biocompatible material conjugated to a ligand and (b) a mammalian cell comprising a molecular circuit comprising: (i) a binding activated transcriptional switch (BTTS) comprising a force-dependent protease cleavage site and a transcriptional activator and (ii) a nucleic acid encoding a payload protein is provided. In these embodiments, binding of the cell of (b) to the ligand of (a) via the BTTS induces proteolytic cleavage of the force-dependent cleavage sites to release the transcriptional activator and induce expression of the payload protein.

In some embodiments, the BTTS may bind directly to the ligand on the material. In other embodiments, the BTTS may bind indirectly to the ligand on the material. In the latter embodiments, the BTTS may bind a molecule that is in solution, where the molecule is sandwiched by the ligand and BTTS (i.e., the ligand and BTTS both bind to the molecule). Soluble factors include secreted proteins that may be found in vivo, for example, in some cases locally to the site of the implant. These embodiments provide a way to respond to soluble cues. As such, binding of the cell of (b) to the ligand of (a) may be direct or indirect (via a soluble factor). In indirect embodiments, the ligand could be another antibody binding domain. In these embodiments, the binding domain on the BTTS and the binding domain of the ligand may recognize different sites in a soluble protein. In some embodiments, binding of the cell of (b) to the ligand of (a) is via a direct interaction between the BTTS and the ligand. In other embodiments, binding of the cell of (b) to the ligand of (a) is via an indirect interaction between the BTTS and the ligand, via a soluble factor to which both the ligand and BTTS bind.

In some embodiments, the mammalian cell may be a stem cell, e.g., a pluripotent stem cell such as an embryonic stem cell (ESC) or an induced pluripotent stem cell (iPSC), or an adult stem cell. Suitable cells include a stem cell (e.g., an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Embryonic Stem Cells (ESC) may be derived from inner cell mass (ICM) of pre-implantation blastocyst-stage embryos. These cells can be potentially be used in allogeneic/‘off-the-shelf’ (donor-derived) cell therapies. In some cases, such cells may be modified for immune compatibility (primarily deletion/disruption of HLA-I and HLA-II classes) (see Kim Stem Cell Reviews and Reports 17 (2021): 1053-1067.

Induced Pluripotent Stem Cells (iPSCs) may be derived from at least skin, lung, heart, stomach, brain, liver, blood, kidney or muscle cells, where stemness and pluripotency can be induced by expression of Yamanaka factors (Oct 3/4, Sox2, Klf4, and c-Myc) among others. These cells can generally be used for autologous (self-derived) or allogeneic (donor-derived) cell therapies. See WO 2010/017562A2 and Liiu, Gele, et al. “Advances in pluripotent stem cells: history, mechanisms, technologies, and applications.” Stem cell reviews and reports 16 (2020): 3-32.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell. Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), intestinal stem cells, hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), endothelial stem cells, neural stem cells, olfactory adult stem cells, neural crest stem cells, testicular cells and skin-derived precursors (SKPs) may be used.

The identity of the payload may vary depending on how the cell is going to be used. In some embodiments, the payload may be cell fate regulator, e.g., a morphogen, one or more transcription factors that induce cell differentiation or a cell surface ligand that induces differentiation of another cell.

Morphogens are signaling molecules that emanate from a restricted region of a tissue and spread away from their source to form a concentration gradient. As the fate of each cell in the field depends on the concentration of the morphogen signal, the gradient prefigures the pattern of development. Mammalian morphogens include Hh (Hedgehog), Wnt, TGF-β (Transforming growth factor β) and BMP (Bone morphogenetic protein), although others are known.

Transcription factors that induce cell differentiation have been described in a variety of publications including: (1) Oh, Yujeong, and Jiwon Jang. “Directed differentiation of pluripotent stem cells by transcription factors.” Molecules and cells 42.3 (2019): 200; (2) Joung, Julia, et al. “A transcription factor atlas of directed differentiation.” Cell 186.1 (2023): 209-229; (3) Smela, Merrick D, Pierson, et al. “Directed differentiation of human iPSCs to functional ovarian granulosa-like cells via transcription factor overexpression.” Elife 12 (2023): e83291; (4) Yamamizu, Kohei, et. al. “Identification of transcription factors for lineage-specific ESC differentiation.” Stem cell reports 1.6 (2013): 545-559; and (5) Van Haute, Lindsey, et al. “Generation of lung epithelial-like tissue from human embryonic stem cells.” Respiratory research 10 (2009): 1-13. Suitable cells (iPSCs or ESC etc.) that differentiate into the cells listed in the following table by expression of the listed transcription factor can be selected from the literature. In some cases, the following transcription factors cant be combined to) make a particular cell type. For example, skeletal muscle can potentially be made by expressing MYODI BAF60C2 and JMJD3 alone or any combination, in a suitable cell type.

| TRANSCRIPTION FACTOR | CELL/TISSUE TYPE |
|---|---|
| NEUROG1 | Neuron |
| NEUROG2 | Neuron |
| NEUROG3 | Neuron |
| NEUROD1 | Neuron |
| NEUROD2 | Neuron |
| BRN2 | Neuron |
| ASCL1 | Neuron |
| MYT1L | Neuron |
| SATB2 | Excitatory neuron |
| FEZF2 | Excitatory neuron |
| DLX2 | GABAergic neuron |
| LHX6 | GABAergic neuron |
| NURR1 | Dopaminergic neuron |
| LMX1A | Dopaminergic neuron |
| ATOH1 | Dopaminergic neuron |
| ISL1 | Motor neuron, Cardiomyocyte |
| LHX3 | Motor neuron |
| MYOD1 | Myocyte/Skeletal Muscle |
| BAF60C2 | Myocyte/Skeletal Muscle |
| JMJD3 | Myocyte/Skeletal Muscle |
| PAX3 | Skeletal myogenic progenitor |
| PAX7 | Skeletal myogenic progenitor |
| GATA4 | Cardiomyocyte |
| MEF2C | Cardiomyocyte |
| TBX5 | Cardiomyocyte |
| ESRRG | Cardiomyocyte |
| MESP1 | Cardiomyocyte |
| SHOX2 | Cardiac pacemaker cell |
| PAX4 | Insulin-producing cell |
| PDX1 | Insulin-producing cell |
| NKX6.1 | Insulin-producing cell |
| NKX2.2 | Insulin-producing cell |
| HEX | Hepatocyte |
| SOX17 | Hepatocyte |
| HNF1A | Hepatocyte |
| HNF4A | Hepatocyte |
| FOXA1 | Hepatocyte |
| FOXA2 | Hepatocyte |
| FOXA3 | Hepatocyte |
| CEBPA | Hepatocyte |
| CEBPB | Hepatocyte |
| GATA2 | Hepatocyte |
| GATA3 | Hepatocyte |
| FERD3L | Neuron |
| LMX1B | Neuron |
| TBXT | Smooth muscle |
| MSGN1 | Smooth muscle |
| ZBTB7B | Smooth muscle |
| DMRTA2 | Smooth muscle |
| BHLHA9 | Smooth muscle |
| PTF1A | EMT smooth muscle |
| FLI1 | Endothelial |
| CDX1 | Mctancphric |
| CDX2 | Metanephric |
| KLF4 | Intestinal epithelial |
| HNF4A | Intestinal epithelial |
| NR5A2 | Intestinal epithelial |
| NHLH1 | Lung ciliated epithelial |
| ASCL2 | Lung ciliated epithelial |
| GRHL3 | trophoblast |
| NR5A1 | Ovarian granulosa-like |
| RUNX1 | Ovarian granulosa-like |
| RUNX2 | Ovarian granulosa-like |
| NKX2.1 | Lung epithelial-like |
| MEF2C | Skeletal muscle |
| TCF3 | Skeletal muscle |
| ESX1 | Skeletal muscle |
| FOXA1 | Liver |
| GBX2 | Liver |
| MYC | Blood cell |
| ELF5 | Blood cell |
| IRF2 | Blood cell |
| ELF1 | Blood cell |
| SFPI1 | Blood cell |
| TGIF1 | Blood cell |
| ETS1 | Blood cell |
| SMAD7 | Neuronal |
| NR2F1 | Neuronal |
| SOX11 | Neuronal |
| DMRT1 | Neuronal |
| SOX9 | Neuronal |
| FOXG1 | Neuronal |
| KLF3 | Neuronal |
| POU5F1 | Neuronal |
| SOX2 | Neuronal |
| ASCL1 | Neuronal |

For example, the following cell fate regulators can be used individually or in combination in the following cells:
hPSCs (ESC or iPSC): neural differentiation via combination of Brn2+Ascl1+Myt1l; NeuroD1; Ngn1; Ngn2
hPSC: cardiomyocyte differentiation: GATA4, MEF2C, Nkx2-5, Activin A, BMP4
hPSC: neural tube/floor plate: SHH
hPSC: gastrula: BMP4; Nodal; WNT3A
hPSC microglia: combination of MAFB, CEPBa, IRF8, PU.1, CEBPb, IRF5
Pancreatic exocrine cells, hepatocytes, or hPSCs to insulin-producing cells: PDX1, NEUROG3, MAFA
hMSC (mesenchymal stem cell): myogenic differentiation (myod1); osteogenic differentiation (bmp2, runx2); chondrogenic differentiation (TGFb3, SOX9).

The following transcription factors may be used in wound repair:

| Transcription Factor | Function/Activity |
|---|---|
| AP-1 | Wound repair/re-epithelialization |
| JUN | Wound repair/re-epithelialization |
| MYC | Wound repair/re-epithelialization |
| E2F-1 | Wound repair/re-epithelialization |
| EGR-1 | Wound repair/re- |
| | epithelialization/angiogenesis |
| GRHL3 | Wound repair/re-epithelialization |
| HOXA3 | Wound repair/re- |
| | epithelialization/angiogenesis |
| HOXD3 | Wound repair/re- |
| | epithelialization/angiogenesis |
| PPARβ/δ | Wound repair/re-epithelialization |
| STAT3 | Wound repair/re-epithelialization |
| CARP | Wound repair/angiogenesis |
| HIF-1α | Wound repair/angiogenesis |

See Schäfer, Matthias, and Sabine Werner. “Transcriptional control of wound repair.” Annu. Rev. Cell Dev. Biol. 23 (2007): 69-92.

If more than one payload needs to be expressed, they can be expressed using separate expression cassettes (all of which may be activated by the BTTS), or a single expression cassette. In the latter embodiment, the coding sequences may be separated by an IRES or a protease cleavage site may be used.

In some embodiments, the cell may additionally secrete a growth factor, e.g., VEGF (Vascular endothelial growth factor), EGF (Epidermal growth factor), FGF (Fibroblast growth factor), TNF-α (Tumor necrosis factor α) or PDGF (Platelet derived growth factor), or a synthetic form thereof, if desired.

The material may be a hydrogel, a polymeric scaffold, collagen, a metal implant, or a phosphate cement. For example, the material can be a PEG-based hydrogel, a gelatin-based hydrogels, a collagen sponge, a hyaluronic acid-based hydrogel, a polymeric scaffold (such as polycaprolactone (PCL), poly-lactic/poly-lactic-co-glycolic acid (PLA/PLGA)), a gold or titanium implants, or a porous or non-porous phosphate cement. Materials can be selected from the following tables:

| Material | Type | Applications | Reference |
|---|---|---|---|
| Poly(acrylic acid) | Polymeric | Wound dressing | (1) |
| grafted bacterial | film | | |
| cellulose | | | |
| Chitosan and konjac | Polymeric | Drug-delivery and | (1) |
| glucomannan | film | wound healing | |
| Alginate | Polymeric | Drug-delivery and | (1) |
| | film | wound healing | |
| Sodium alginate, | Polymeric | Wound dressing | (1) |
| gelatin, and reduced | film | | |
| graphene oxide | | | |
| Agarose and chitosan | Polymeric | Scaffold, tissue | (1) |
| | sponge | engineering | |
| Gelatin | Polymeric | Scaffold, tissue | (1) |
| | sponge | engineering | |
| Fibroin, chitin, silver | Polymeric | Scaffold, | (1) |
| nanoparticles | sponge | antibacterial | |
| Collagen and ZnO | Polymeric | Scaffold, wound | (1) |
| particles | sponge | dressing | |
| Gelatin and Polyvinyl | Polymeric | Scaffold, skin | (1) |
| alcohol (PVA) | sponge | regeneration | |
| Collagen and | Polymeric | Scaffold, bone- | (1) |
| hydroxyapatite | sponge | tissue engineering | |
| Chitosan | Polymeric | Scaffold, bone- | (1) |
| | sponge | tissue engineering | |
| Chitosan cross-linked | Hydrogel | Vascular stents | (1) |
| with epoxy polymers | | | |
| Alginate-gellan gum | Hydrogel | 3D-printer | (1) |
| | | scaffold | |
| Collagen | Hydrogel | Corneal/Cardiac | (1) |
| | | tissue engineering | |
| Poly(ε-caprolactone)- | Hydrogel | Corneal tissue | (1) |
| poly(ethylene glycol) | | engineering | |
| and gelatin | | | |
| methacrylate | | | |
| Collagen/polyacrylamide | Hydrogel | Wound dressing | (1) |
| Collagen/hyaluronic acid | Hydrogel | Bone tissue | (1) |
| | | engineering | |
| Sodium alginate, | Hydrogel | Wound dressing | (1) |
| Bioglass, poly(lactic- | | | |
| co-glycolic acid) | | | |
| Carbon nanotube | Injectable | Haemorrhage | (1) |
| (CNT) and glycidyl | hydrogel | homeostasis and | |
| methacrylate | | wound healing | |
| functionalized | | | |
| quaternized chitosan | | | |
| Collagen I and III | Injectable | Skin tissue | (1) |
| | hydrogel | engineering | |
| Gelatin/hyaluronic | Injectable | Cardiac tissue | (1) |
| acid | hydrogel | engineering | |
| Gelatin gold | Injectable | Bone tissue | (1) |
| nanoparticles | hydrogel | engineering | |
| PLA, calomer | Thermoplastics | Heat triggered | (1) |
| | | vascular stent | |
| MM5520 and MM6520 | Thermoplastics | Light triggered | (1) |
| | | vascular stent | |
| Cu-clad polyimide | Thermoplastics | Wireless actuated | (1) |
| | | Shape-memory | |
| | | plastic for drug | |
| | | delivery | |
| Polyurethane | Thermoplastics | Shape memory | (1) |
| | | scaffold | |
| Acrylonitrile butadiene | Thermoplastics | 3D printed | (1) |
| styrene (ABS) and | | scaffold | |
| polylactic acid (PLA) | | | |
| POROLAY | Thermoplastics | 3D printed | (1) |
| | | scaffold drug | |
| | | delivery | |
| Aldehyde modified | Hydrogel | Bio-responsive | (2) |
| hyaluronic acid and | | | |
| dextran sulfate | | | |
| Poly(β-amino ester) | Hydrogel | Ph-responsive | (3) |

| Polymer | Type | Reference |
|---|---|---|
| Collagen | Natural polymer | (1) |
| Fibrin | Natural polymer | (1) |
| Keratin | Natural polymer | (1) |
| Fibronectin | Natural polymer | (1) |
| Laminin | Natural polymer | (1) |
| Alginate | Natural polymer | (1) |
| Chitin | Natural polymer | (1) |
| Chitosan | Natural polymer | (1) |
| Polylactic acid (PLA) | Synthetic polymer | (1) |
| Polyvinyl alcohol (PVA) | Synthetic polymer | (1) |
| Polycaprolactone (PCL) | Synthetic polymer | (1) |
| Polyethylene glycol (PEG) | Synthetic polymer | (1) |
| Polylactic-co-glycolic acid | Synthetic polymer | (1) |
| (PLGA) | | |
| Gelatin methacrylate | Synthetic polymer | (1) |
| Glycol-chitosan | Synthetic polymer | (1) |
| Poly(N-isopropylacrylamide) | Synthetic polymer | (1) |
| Carboxymethylcellulose | Synthetic polymer | (1) |
| | Synthetic polymer | (1) |

See:
(1) Kalirajan C, Dukle A, Nathanael A J, Oh T H. Manivasagam G. A Critical Review on Polymeric Biomaterials for Biomedical Applications. Polymers (Basel). 2021 Sep. 6; 13(17):3015. PMID: 314503054; PMCID: PMC8433665.
(2) Purcell, Brendan P., et al. “Injectable and bioresponsive hydrogels for on-demand matrix metalloproteinase inhibition.” Nature materials 13.6 (2014): 653-661.
(3) Zhang, Yanfeng, et al. “Trigger-responsive poly (β-amino ester) hydrogels.” ACS Macro Letters 3.7 (2014): 693-697?.

The ligand that is conjugated to the material should not be endogenous to the same species as the cell. For example, if the cell is human then the ligand does not need to be a human antigen. In some embodiments, a non-immunogenic ligand may be used.

The ligand can be conjugated to the material by any suitable method. In some embodiments, the material may be treated so that it has reactive groups e.g., a hydroxyl, sulfhydryl, carboxy or amine group, to which the ligand can be conjugated, either covalently or non-covalently.

Conjugation methods are known. For example, the material can be modified to comprise a variety of reactive groups, including, but not limited to, N-hydroxysuccinimidyl ester, sulfo-N-hydroxysuccinimidyl ester, a halo-substituted phenol ester, pentafluorophenol ester, a nitro-substituted phenol ester, an anhydride, isocyanate, isothiocyanate, an imidoester, maleimide, iodoacetyl, hydrazide, an aldehyde, or an epoxide. Other suitable groups are known in the art and may be described in, e.g., Hermanson, “Bioconjugate Techniques” Academic Press, 2nd Ed., 2008. The ligand can be produced separately and then conjugated to the reactive group using known chemistry. The ligand may additionally contain a spacer in some cases. In some embodiments, the ligand may be attached to the material via Click chemistry.

In some embodiments, the material may be additionally coated in a non-specific cell adhesive, e.g., a polysaccharide such as glycosaminoglycan, which should allow cells to ‘stick’ to the material, if necessary.

The BTTS is a cleavable fusion protein that contains: (a) an extracellular binding domain comprising a protein binding domain (e.g., scFv or nanobody) that binds to the ligand, (b) a transmembrane domain, (c) one or more force-dependent cleavage sites, and (d) an intracellular domain comprising a transcriptional activator, where binding of the binding domain to the ligand on the material induces proteolytic cleavage of the one or more force-dependent cleavage sites to release the transcriptional activator. In any embodiment, the BTTS may additionally comprise a force sensing region.

In this switch, the fusion protein is cleaved to release the intracellular domain when the extracellular domain of the fusion protein engages with the ligand on the material. As such, in many cases, the fusion protein may contain a force sensing region (which is typically in the extracellular domain) and one or more force-dependent cleavage sites that are cleaved when the force sensing region is activated. The position of the force-dependent cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the force sensing region and/or the one or more force-dependent cleavage sites may be from a Delta/Serrate/Lag2 (DSL) superfamily protein, as reviewed by Pintar et al (Biology Direct 2007 2: 1-13). For example, the force sensing region and/or the one or more force-dependent cleavage sites may be from Notch (see Morsut Cell. 2016 164: 780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (IL1R2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15: 849-857), a trans-membrane segment, and a relatively short (~100-150 amino acids) intracellular domain. These sequences permit the binding-triggered release of a transcriptional activator from the membrane in their natural environment and can be readily adapted herein.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In some cases, the shear force generated by binding of the extracellular domain of this fusion protein to another cells unfolds the force sensing region (which, in the case of Notch contains EGF-like repeats whereas in other protein is made up of other sequences such as the A2 domain in vWF (see, e.g., J Thromb Haemost. 2009 7:2096-105, Lippok Biophys J. 2016 110: 545-54, Lynch Blood. 2014 123: 2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut et al, Cell. 2016 164: 780-91, WO2016138034 and WO2019099689, among other places).

In some cases, the fusion protein includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO:1), where cleavage occurs between the “RE” sequence. As another example, an amino acid sequence comprising an S1 ligand-inducible proteolytic cleavage site can have the amino acid sequence RQRRELDPM (SEQ ID NO:2), where cleavage occurs between the “RE” sequence.

In some cases, the fusion protein polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO:3), where cleavage occurs between the “AV” sequence. As another example, an amino acid sequence comprising an S2 ligand-inducible proteolytic cleavage site can have the amino acid sequence KIEAVQSE (SEQ ID NO:4), where cleavage occurs between the “AV” sequence.

In some cases, the fusion protein includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO:5), where cleavage occurs between the “GV” sequence. In some cases, an S3 ligand-inducible proteolytic cleavage site comprises the amino acid sequence GCGVLLS (SEQ ID NO:6).

In some cases, the fusion protein polypeptide lacks an S1 ligand-inducible proteolytic cleavage site. In some cases, the BTTS lacks an S2 ligand-inducible proteolytic cleavage site. In some cases, the BTTS lacks an S3 ligand-inducible proteolytic cleavage site. In some cases, the BTTS lacks both an S1 ligand-inducible proteolytic cleavage site and an S2 ligand-inducible proteolytic cleavage site. In some cases, the BTTS includes an S3 ligand-inducible proteolytic cleavage site; and lacks both an S1 ligand-inducible proteolytic cleavage site and an S2 ligand-inducible proteolytic cleavage site.

In some embodiments, the fusion protein may have an vWF A2 sequence or a variation thereof, an ADAMTS13 cleavage site (which may be described by the consensus sequence HEXXHXXGXXHD; Crawley, Blood. 2011 118:3212-21), and an S3 or 7-secretase cleavage site, although many other arrangements exist. In some embodiments, the switch may contain components that are borrowed from Notch. In other embodiments, the switch may not contain components that are from Notch.

For simplicity, BTTSs, including but not limited to chimeric notch receptor polypeptides, are primarily single polypeptide chains. However, BTTSs, including chimeric notch receptor polypeptides, may be divided or split across two or more separate polypeptide chains where the joining of the two or more polypeptide chains to form a functional BTTS, e.g., a chimeric notch receptor polypeptide, may be constitutive or conditionally controlled. For example, constitutive joining of two portions of a split BTTS may be achieved by inserting a constitutive heterodimerization domain between the first and second portions of the split polypeptide such that upon heterodimerization the split portions are functionally joined.

Useful BTTSs that may be employed in the subject methods include, but are not limited to modular extracellular sensor architecture (MESA) polypeptides. A MESA polypeptide comprises: a) a ligand binding domain; b) a transmembrane domain; c) a protease cleavage site; and d) a functional domain. The functional domain can be a transcription regulator (e.g., a transcription activator, a transcription repressor). In some cases, a MESA receptor comprises two polypeptide chains. In some cases, a MESA receptor comprises a single polypeptide chain. Non-limiting examples of MESA polypeptides are described in, e.g., U.S. Patent Publication No. 2014/0234851; the disclosure of which is incorporated herein by reference in its entirety.

Useful BTTSs that may be employed in the subject methods include, but are not limited to polypeptides employed in the TANGO assay. The subject TANGO assay employs a TANGO polypeptide that is a heterodimer in which a first polypeptide comprises a tobacco etch virus (Tev) protease and a second polypeptide comprises a Tev proteolytic cleavage site (PCS) fused to a transcription factor. When the two polypeptides are in proximity to one another, which proximity is mediated by a native protein-protein interaction, Tev cleaves the PCS to release the transcription factor. Non-limiting examples of TANGO polypeptides are described in, e.g., Barnea et al. (Proc Natl Acad Sci USA. 2008 Jan. 8; 105(1):64-9); the disclosure of which is incorporated herein by reference in its entirety.

Useful BTTSs that may be employed in the subject methods include, but are not limited to von Willebrand Factor (vWF) cleavage domain-based BTTSs, such as but not limited to e.g., those containing a unmodified or modified vWF A2 domain. A subject vWF cleavage domain-based BTTS will generally include: an extracellular domain comprising a first member of a binding pair; a von Willebrand Factor (vWF) cleavage domain comprising a proteolytic cleavage site; a cleavable transmembrane domain and an intracellular domain. Non-limiting examples of vWF cleavage domains and vWF cleavage domain-based BTTSs are described in Langridge & Struhl (Cell (2017) 171(6):1383-1396); the disclosure of which is incorporated herein by reference in its entirety.

Useful BTTSs that may be employed in the subject methods include, but are not limited to chimeric Notch receptor polypeptides, such as but not limited to e.g., synNotch polypeptides, non-limiting examples of which are described in PCT Pub. No. WO 2016/138034, U.S. Pat. Nos. 9,670,281, 9,834,608, Roybal et al. Cell (2016) 167(2):419-432, Roybal et al. Cell (2016) 164(4):770-9, and Morsut et al. Cell (2016) 164(4):780-91; the disclosures of which are incorporated herein by reference in their entirety. The “SNIPR” switch is another example of a BTTS (Zhu et al 2022 Cell. 185: 1431-1443 and WO2021061856), although others (including the A2, MESA, Tango, ChaCha, RASER, Cal-Light switches.

Expression of the BTTS in the cell may be constitutive or inducible, e.g., by binding of another BTTS to another ligand (a second ligand), which may be on the same material.

Examples of transcriptional activators that can be part of the fusion protein are numerous and include artificial transcription factors (ATFs) such as, e.g., Zinc-finger-based artificial transcription factors (including e.g., those described in Sera T. Adv Drug Deliv Rev. 2009 61(7-8):513-26; Collins et al. Curr Opin Biotechnol. 2003 14(4):371-8; Onori et al. BMC Mol Biol. 2013 14:3. In some cases, the transcriptional activator may contain a GAL4 DNA binding domain, which binds to the Gal4 responsive UAS, which has been well characterized in the art. Examples of suitable transcriptional activators include GAL4-VP16 and GAL4-VP64, although many others could be used. As would be appreciated, the identity of the transcription activators may vary. In some embodiments, the transcription factor may have a DNA binding domain that binds to a corresponding promoter sequence and an activation domain. In many embodiments, the DNA binding domain transcription factor may be independently selected from Gal4-, LexA, Tet-, Lac-, dCas9-, zinc-finger- and TALE-based transcription factors. TALE- and CRISPR/dCas9-based transcription factors are described in Lebar (Methods Mol Biol. 2018 1772: 191-203), among others. The binding sites for such domains are well known or can be designed at will. The transcription factors can have any suitable activation domain, e.g., VP16, VP64, Ela, Sp1, VP16, CTF, GAL4 among many others.

The extracellular binding domain of the BTTS can be the binding domain of an antibody, e.g., a scFv or nanobody, as described above. As would be apparent, the extracellular binding domain of the BTTS should not bind to other cell surface antigens in the species in which the cell is being used.

In some embodiments, the BTTS comprises i. an extracellular binding domain that binds to the ligand (e.g., a scFv or nanobody) ii. an optional force sensing region, iii. a transmembrane domain, iv. a force-dependent cleavage sites and v. an intracellular domain comprising a transcriptional activator, where binding of the extracellular binding domain to the ligand induces proteolytic cleavage of the force-dependent cleavage site to release the transcriptional activator. In some embodiments, the mammalian cell may be a stem cell, the payload protein is a cell fate regulator, and expression of the payload protein causes the stem cell to differentiate.

### Methods

The above-described system may be used in a variety of methods. In some embodiments, the method may comprise obtaining the components of the system (e.g., the material) and the cell, and contacting them together to induce expression of the payload. In some embodiments, the mammalian cell is a stem cell, the payload protein is a cell fate regulator, and expression of the payload protein causes the stem cell to differentiate. The contacting may be done in vitro, ex vivo or in vivo. In some embodiments, the contacting may be done in presence of a second cell and wherein the payload protein induces differentiation of the second cell (e.g., via expression of a morphogen or cell surface ligand). The second cell may be endogenous to the subject, if this method is practiced in vivo (e.g., the second cell may be a stem cell in the subject).

In any embodiment, the method may comprise: (i) administering (a) to one or more sites in the mammal; and (ii) administering (b) to the mammal, thereby inducing the payload at the one or more sites of (a). In some embodiments, the one or more sites may be internal to (i.e., beneath the skin of) the subject. In other embodiments the one or more sites may be on the skin of the subject. In these embodiments, the cell may be administered systemically (e.g., by infusion). However, in some embodiments, the cell may be administered locally to site of the material.

Also included is a method comprising administering a mammalian cell (as described above) to a subject. In these embodiments, the cell may comprise a molecular circuit, the circuit comprising: (i) a binding activated transcriptional switch (BTTS) comprising a force-dependent protease cleavage site and a transcriptional activator; and (ii) a nucleic acid encoding a payload protein, and (b) the subject comprises a biocompatible material conjugated to a ligand. In these embodiments, binding of the cell to the ligand via the BTTS induces proteolytic cleavage of the force-dependent cleavage sites to release the transcriptional activator and induce expression of the payload protein. In some embodiments, the material may be implanted in the subject, beneath the skin. In some embodiments, the method may involve involving surgically implanting the material and then, after a period of time (e.g., at least 1 day, at least 1 week or at least 1 month) administering the cell to the subject.

The present method may be used in a variety of treatments, e.g., in a variety of wound repair, tissue rejuvenation, tissue regeneration. In some embodiments, the method may be used for tissue regeneration in vivo. In these embodiments, the material may be placed in or on the subject, and the cells may be administered to the subject. After making contact with the material, the cell may differentiate into a cell type that is suitable for tissue regeneration. This embodiment could be used for wound repair or the treatment of a burn or another injury and may be used as a combination treatment with a skin graft or wound dressing, for example. The present method can be used to treat cardiovascular disease, cell deficiencies, etc., or for bone, corneal, vascular, muscle or skin regeneration, as needed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

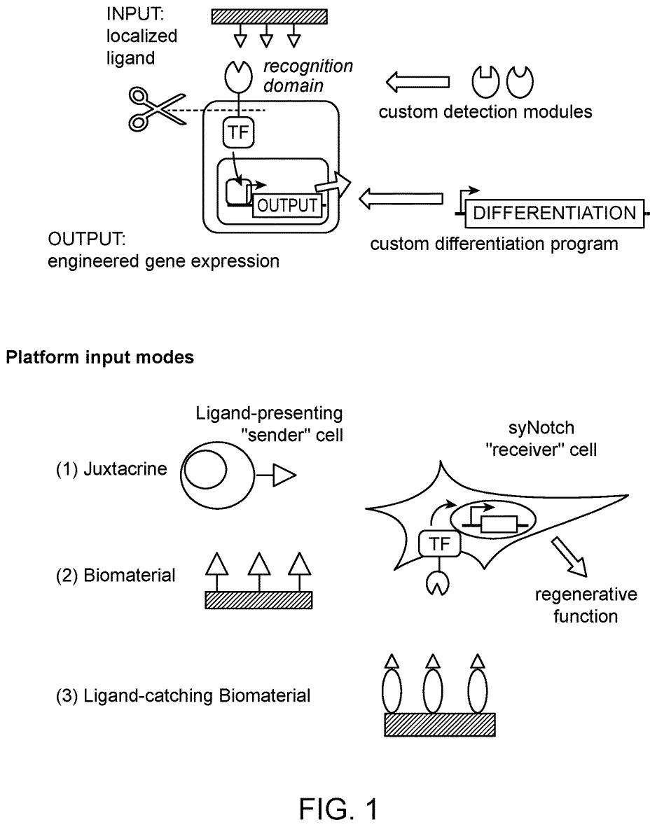

Attempts to produce cell-based therapies for regeneration and repair have often failed because of the lack of an ability to direct cellular responses such as differentiation in a spatially precise way. Provided herein is a solution to this problem by engineering complementary material-cell pairs: biomaterials are engineered with orthogonal ligands that are specifically recognized by complementary engineered responder cells. In this case, responder cells are engineered with synthetic Notch (synNotch) receptors (or any functionally analogous binding triggered transcriptional switches), which are activated by cognate ligands incorporated into the complementary biomaterial (FIG. 1). The input ligands can be orthogonal to other biological interactions, thus allowing the material to only generate a response in the engineered responder cells. This type of engineered material/cell platform can be used to locally drive processes such as stem cell differentiation or the production of morphogens.

Here, a means whereby cellular responses to biomaterials can be custom defined by combining engineered materials with cells expressing cognate synthetic biology control modules is presented. Such privileged channels of material-to-cell communication can govern pluripotent stem cell differentiation in response to materials decorated with otherwise bioinert ligands. Further, it has been show that engineered cellular behaviors are confined to programmed biomaterial surfaces, highlighting the potential to use this platforn to spatially organize cellular responses to bulk soluble factors. This integrated approach of co-engineering cells and biomaterials for orthogonal interactions opens new avenues for reproducible control of cell-based therapies and tissue replacements.

In the experiments outlined here, gene circuits encoding synNotch platform elements were delivered to target cells (murine and human embryonic stem cells and murine L929 fibroblasts) by lentiviral transduction. SynNotch ligand or anti-ligand nanobodies capable of immobilizing soluble ligand in culture medium were conjugated to biomaterials via primary amine chemistry, offering the opportunity for materials to serve as a new input mode via synNotch (FIG. 1). SynNotch activation was monitored by mCherry expression, and the ability of synNotch to drive differentiation of stem cells was assessed through immunolabeling, assayed via flow cytometry or confocal microscopy.

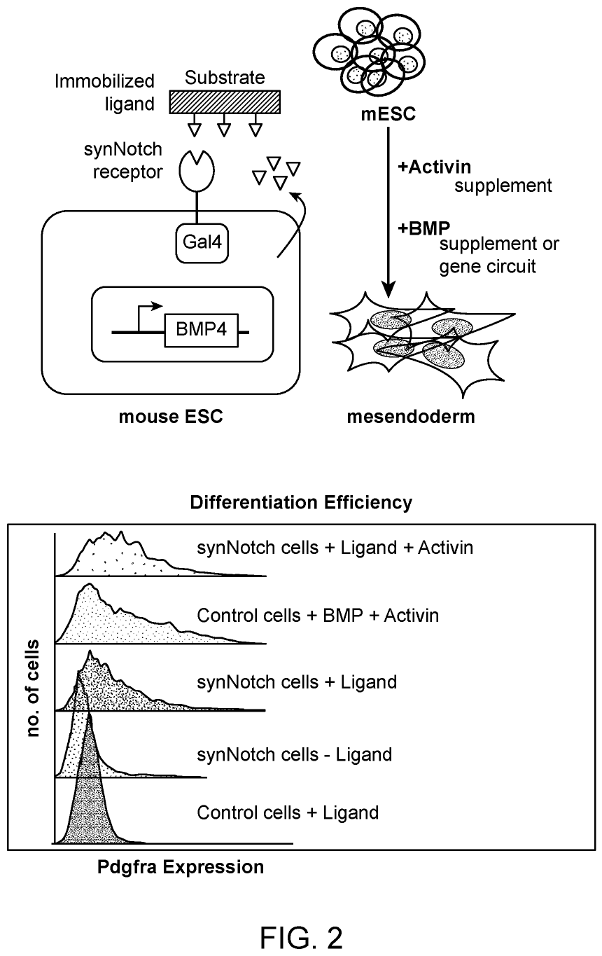

It has been shown that biomaterials programmed with synNotch ligand activate synNotch can be used to induce morphogen (e.g., BMP4) expression in mESCs, leading to mesodermal differentiation as indicated by activation of Flk1 and Pdgfra expression reporters (FIG. 2).

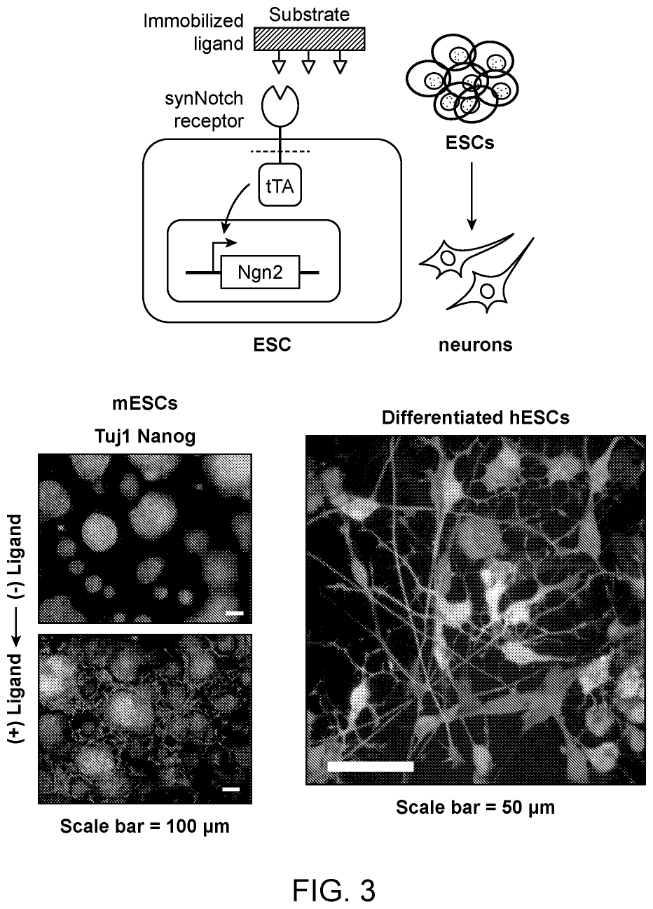

In parallel experiments, it has been shown that materials displaying synNotch ligand can induce neuronal differentiation of ESCs engineered to express Neurogenin2 upon synNotch activation (FIG. 3). This was observed by loss of pluripotency, combined with neurite projection and neuron-specific tubulin (Tuj1) expression.

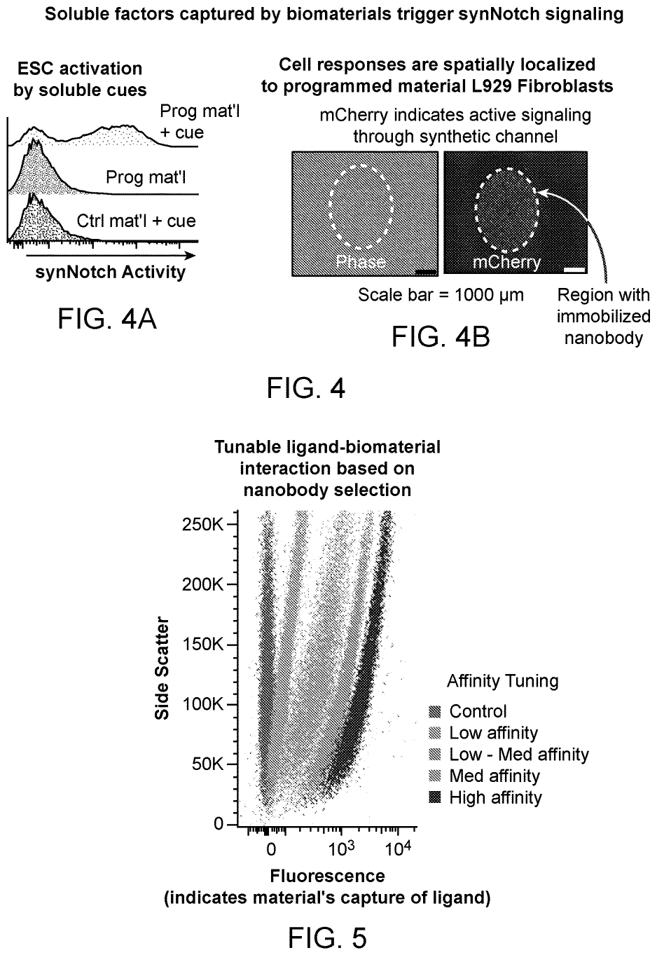

Programming biomaterials with an anti-ligand nanobody (e.g., GFP-TRAP nanobody for capturing GFP ligand) converts the juxtacrine synNotch platform to a paracrine platform capable of detecting soluble cues. Embryonic stem cells cultured on such programmed materials activate potently in response to GFP ligand addition to the bulk, soluble medium (FIG. 4A). Further, by selectively programming specific regions of materials to bind ligand, one can spatially coordinate responses to globally present, soluble factors (FIG. 4B). It has also been demonstrate that one can tune biomaterial binding to soluble factors, a feature that will likely lead to dynamic readouts of the cellular microenvironment based on engineered features of cell-biomaterial interactions. FIG. 5 shows that the system can be tuned.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A reagent system comprising:
(a) a biocompatible material conjugated to a ligand; and
(b) a mammalian cell comprising a molecular circuit, the circuit comprising:
(i) a binding activated transcriptional switch (BTTS) comprising a force-dependent protease cleavage site and a transcriptional activator; and
(ii) a nucleic acid encoding a payload protein,
wherein binding of the cell of (b) to the ligand of (a) via the BTTS induces proteolytic cleavage of the force-dependent cleavage sites to release the transcriptional activator and induce expression of the payload protein.

2. The system of claim 1, wherein the mammalian cell is a stem cell.

3. The system of claim 2, wherein the stem cell is a pluripotent stem cell.

4. The system of claim 2, wherein the stem cell is an adult stem cell.

5. The system of any prior claim, wherein the payload is a cell fate regulator.

6. The system of claim 5, wherein the payload is a morphogen.

7. The system of claim 5, wherein the payload is one or more transcription factors that induce differentiation of the cell.

8. The system of claim 5, wherein the payload is a cell surface ligand that induces differentiation of another cell.

9. The system of any prior claim, wherein the material is a hydrogel, a polymeric scaffold, collagen, a metal implant, or a phosphate cement.

10. The system of any prior claim, wherein the mammalian cell is a stem cell, the payload protein is a cell fate regulator, and expression of the payload protein causes the stem cell to differentiate.

11. The system of any prior claim, wherein the BTTS comprises:
i. an extracellular binding domain that binds to the ligand,
ii. a transmembrane domain,
iii. a force-dependent cleavage site, and
iv. an intracellular domain comprising a transcriptional activator, where binding of the extracellular binding domain to the ligand induces proteolytic cleavage of the force-dependent cleavage site to release the transcriptional activator.

12. The system of any prior claim, wherein the ligand is not found in or on cells of the same species as the mammalian cell.

13. The system of any prior claim, wherein binding of the cell of (b) to the ligand of (a) is via a direct interaction between the BTTS and the ligand.

14. The system of any prior claim, wherein binding of the cell of (b) to the ligand of (a) is via an indirect interaction between the BTTS and the ligand, via a soluble factor to which both the ligand and BTTS bind.

15. A method comprising:
obtaining a system of any of claims 1-14; and
contacting (a) and (b), thereby inducing the payload.

16. The method of claim 15, wherein the mammalian cell is a stem cell, the payload protein is a cell fate regulator, and expression of the payload protein causes the stem cell to differentiate.

17. The method of claim 15, wherein the contacting is done in presence of a second cell and wherein the payload protein induces differentiation of the second cell.

18. The method of any of claims 15-17, wherein the contacting is done in vitro, ex vivo or in vivo.

19. The method of any of claims 15-18, wherein method comprises:
(i) administering (a) to one or more sites in the mammal; and
(ii) administering (b) to the mammal, thereby inducing the payload at the one or more sites of (a).

20. The method of claim 19, wherein the administering of (ii) is systemic administration.

21. The method of claim 19, wherein the mammalian cell is a stem cell, the payload protein is a cell fate regulator, and expression of the payload protein causes the stem cell to differentiate locally at the one or more sites in the mammal.

22. A method comprising:
administering a mammalian cell to a subject, wherein:
(a) the cell comprises a molecular circuit, the circuit comprising:
(i) a binding activated transcriptional switch (BTTS) comprising a force-dependent protease cleavage site and a transcriptional activator; and
(ii) a nucleic acid encoding a payload protein, and
(b) the subject comprises a biocompatible material conjugated to a ligand or ligand capturing domain;
wherein binding of the cell to the ligand via the BTTS induces proteolytic cleavage of the force-dependent cleavage sites to release the transcriptional activator and induce expression of the payload protein.

23. The method of claim 22, wherein the material is implanted in the subject, beneath the skin.
